(19) United States
(12) Patent Application Publication
(10) Pub. No.: US 2010/0191202 A1
(43) Pub. Date: Jul. 29, 2010

(54) CONNECTOR FOR AN OSTOMY APPARATUS, AND OSTOMY APPARATUS INCLUDING SAME

(75) Inventors: Aurélien Hogard, Bayonne (FR); Laurent Lalet, Anglet (FR)

Correspondence Address: RATNERPRESTIA, P.O. BOX 980, VALLEY FORGE, PA, 19482, US

(73) Assignee: B. BRAUN MEDICAL SAS, BOULOGNE-BILLANCOURT (FR)

(21) Appl. No.: 12/531,173

(22) PCT Filed: Mar. 11, 2008
(86) PCT No.: PCT/FR08/50411

§ 371 Date: Mar. 15, 2010

(30) Foreign Application Priority Data
Mar. 15, 2007 (EP) 07 01874

(51) Int. Cl. A61F 5/448

(52) U.S. Cl. 604/342

(57) ABSTRACT

The invention relates to a connector for an ostomy apparatus of the type that includes a first member attached to a holder to be attached to the skin of a user and a second member attached to a bag. The first member defines an opening, is made from a flexible material, and includes a continuous groove emerging radially. The second member includes an elastic ring and a flap that comprises, between the ring and the periphery thereof, a deformable uncoupling area permitting a relative movement of the portions of the flap which are respectively adjacent to the elastic ring and to the periphery.

This invention relates to a connector for an ostomy apparatus and an ostomy apparatus that includes it.

There are many existing types of ostomy apparatus connectors, belonging to two main categories: mechanical connectors and glued connectors. The invention relates to a mechanical connector.

A connector for an ostomy apparatus has two main functions. The first function is to support the weight of the apparatus, particularly when it contains evacuated material, and the second function is to keep liquids and gases (bad odors) from escaping the apparatus. An additional function, often present at the user's request, is a safety feature supplied either by locking the mechanical attachment or by a redundant attachment between the apparatus and its holder to keep it from separating from the holder, even if the main mechanical attachment device fails. This function is carried out, for example, when a noise that is audible to the user is created when the mechanical attachment is engaged.

In most mechanical ostomy apparatus connectors, the main mechanical holder function and the sealing function are carried out by separate members. More specifically, a first member centers a first connector member on another member, and a seal, such as a deformable lip, forms a seal at a different location than the mechanical support. The portions responsible for this mechanical support are not elastically deformable, or only to a lesser extent to allow an elastic engagement, but not to form the seal.

These mechanical connectors, in which the rigid portions cooperate for centering and support, present elastic properties only in the lip, which deforms due to its thinness, even though it generally is not sufficiently flexible for the patient's comfort.

We have already considered ostomy apparatus connectors that have, on a holder attached to the patient, a groove that emerges radially outward and in which an elastic ring is hung. Given the radial direction of the groove, the elastic ring must have a certain range of elastic stretch. However, because this ring is elastic, it is conceivable that, when force is applied by the bag, the elastic ring tends to stretch and separate from the flange that defines the holder's groove. For this reason, as described in the document FR_{—}2 387 643, the elastic ring is used only for a sealing function, blocking evacuated materials and bad odors from passing through, while another device mechanically supports the weight of the bag. In the aforementioned document, this mechanical support device is comprised of a ring that holds a neck formed by the bag onto the periphery of the flange outside the holder attached to the user.

The invention relates to a connector for an ostomy apparatus in which a member's elastic ring is fitted into a groove emerging radially from another member, and the ring is responsible both for sealing in evacuated materials and bad odors and for mechanically supporting the apparatus, even when it is full. This result is achieved by having a device around the elastic ring that applies the weight of the apparatus to the only portion of the elastic ring located above the groove. This upper portion of the ring thus works by compression and performs its sealing and mechanical support functions equally effectively as the weight of the apparatus increases. This device is, for example, a deformable area that relieves the weight of the apparatus from the lower portion of the ring so that the lower portion does not tend to move away from the groove.

More precisely, the invention relates to a connector for an ostomy apparatus of the type that includes a first member attached to a holder to be attached to the skin of a user and a second member attached to a bag that forms a receptacle. According to the invention, the first member defines an opening and is made of a flexible material so that it can adapt to variations in the shape of the part of the user's body to which the holder is attached, and it defines a continuous groove emerging radially around the opening. The second member includes an elastic ring surrounding an opening and a flap on a plane between the elastic ring and the periphery thereof, with the at-rest inside diameter of the second member's elastic ring being less than the diameter of the first member's groove and the flap being designed to be secured to a bag. The second member also includes, between the ring and the flap, a device for applying the weight of the apparatus to the only portion of the elastic ring located above the groove.

Preferably, the device for applying the weight of the apparatus to the only portion of the elastic ring located above the groove is a deformable uncoupling area such that a portion of the flap adjacent to the elastic ring and a portion of the flap adjacent to the periphery allow a relative movement on said plane when the flap is subjected to a force acting on its plane.

In one embodiment, the deformable area is formed all around the ring in each radial direction, and the deformable area has an ability to stretch more than it can contract. In this case, the side of the deformable area with the greatest deformation capacity is preferably located at the bottom on a standing user.

In another embodiment, the deformable area only surrounds a portion of the ring on the side intended to be located at the bottom on a standing user.

Preferably, the second member has at least one traction pin intended to apply a radial outward force to the elastic ring.

Preferably, the pin is at least attached at a location that is closer than the deformable area to the inside of the flap. For example, the traction pin is attached to the elastic ring.

Preferably, the second member includes three traction pins spaced about 13° apart and located opposite of the side intended to be located at the bottom on a standing user.

Preferably, the second member, formed by the elastic ring, the device applying the weight of the apparatus, the flap, and the pins, if applicable, is formed in one piece. For example, the second member is formed in one piece by injection molding.

Preferably, the first member's groove is defined, on the side intended to be farthest from the holder, by an outer flange whose radial dimension on the outside of the opening is greater than the side intended to be located at the bottom on a standing user.

Preferably, the radial dimension of the first member's outer flange is greater at each location that corresponds to a pin on the second member.

Preferably, the first member is made from an elastomer.

Preferably, the connector also includes a redundant member that mechanically attaches the first member to the second member. For example, the redundant member is either a hook and loop attachment or a snap.

The invention also relates to an ostomy apparatus that comprises at least two layers of foil defining a receptacle, such that one foil is attached to a member constituted by a second connector member according to one of the previous paragraphs.

Preferably, the second member is connected to the foil by either soldering or paste.

A first advantage of the connector according to the invention is that it may be less costly because each of the two connector members can easily be formed in one piece by injection molding.

A second advantage of the connector according to the invention is that both connector members can be flexible, thereby providing greater comfort to the user.

A third advantage of the connector according to the invention is that the wide portions of the flange may give the user a sufficient feeling of security, making it possible to remove all redundant connection devices.

Other characteristics and advantages of the invention will be better understood upon reading the following description, with reference to the attached drawings:

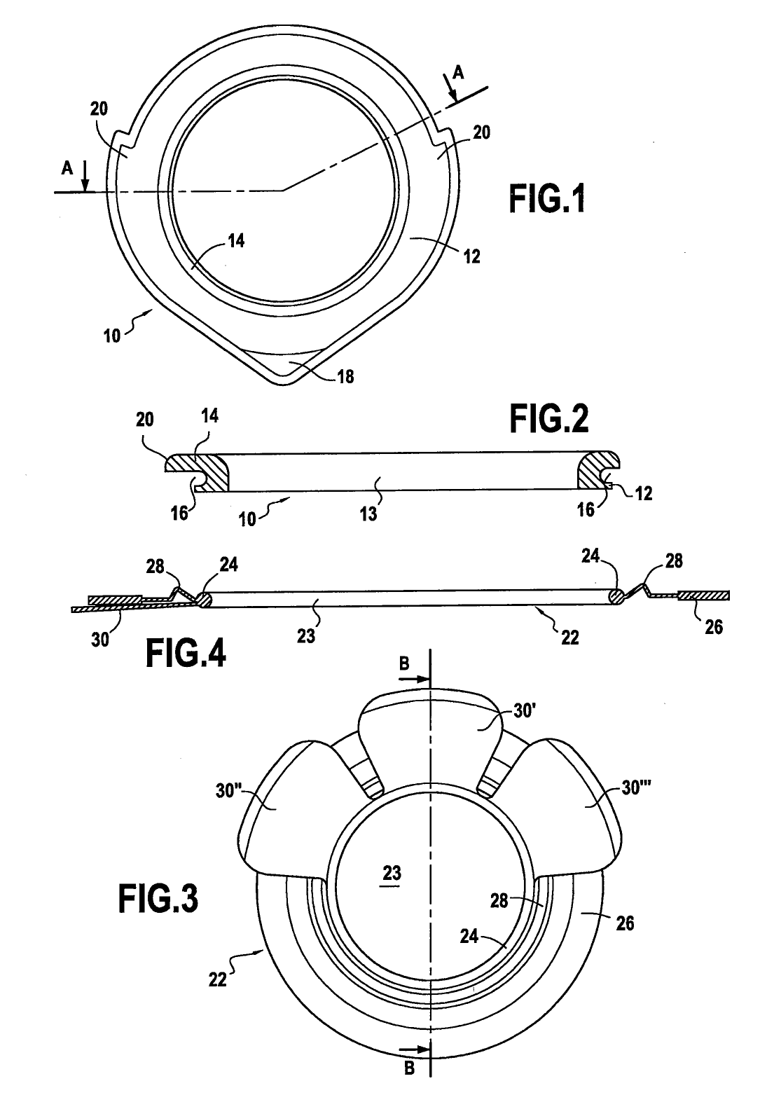

FIG. 1 is an outline sketch of the first member of a connector for an ostomy apparatus according to the invention, intended to be permanently attached to a holder glued to the skin of a user.

FIG. 2 is a section following line A_A in FIG. 1.

FIG. 3 is an outline sketch of a second member of a connector for an ostomy apparatus according to the invention, intended to coordinate with the first member in FIGS. 1 and 2.

FIG. 4 is a section following line B_B in FIG. 3.

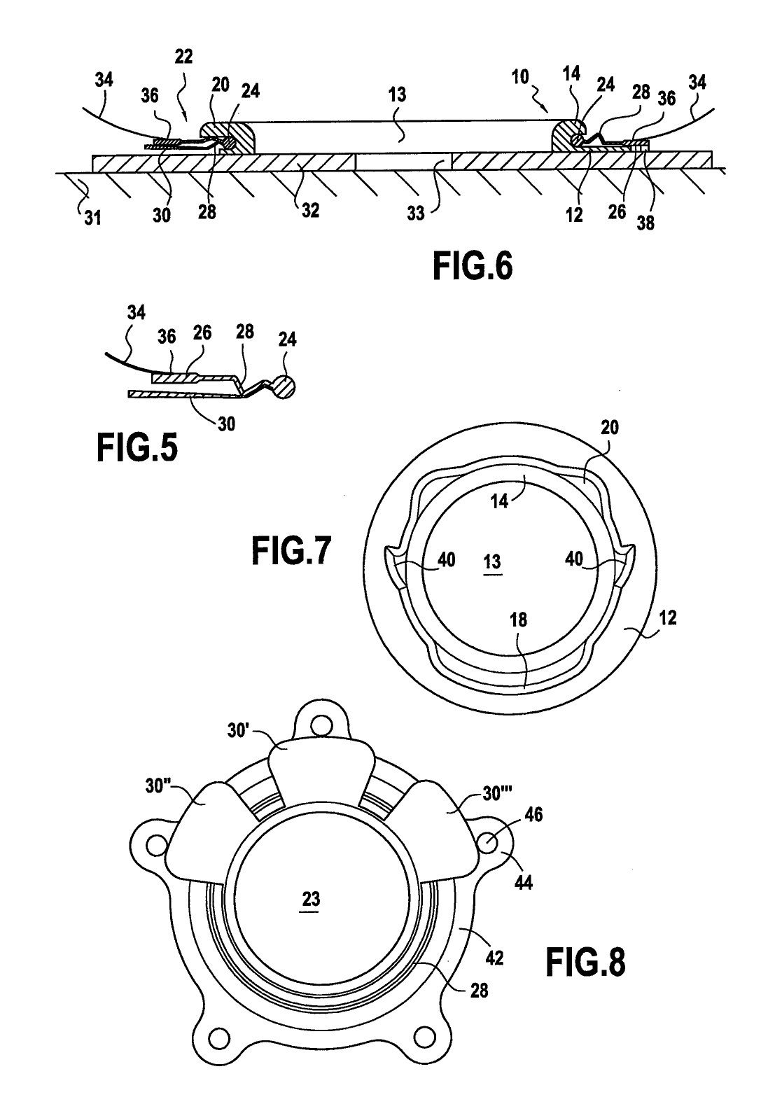

FIG. 5 is an enlargement of the left portion of FIG. 4.

FIG. 6 is a section of a connector for an ostomy apparatus according to the invention in corresponding position, according to the section lines A_A and B_B in FIGS. 1 and 3.

FIG. 7 is similar to FIG. 1, but it shows a variant of the first member of a connector for an ostomy apparatus according to the invention.

FIG. 8 is similar to FIG. 3, but it shows a variant of the second member of a connector for an ostomy apparatus according to the invention.

The connector for an ostomy apparatus according to the invention is intended to be used under the normal conditions of use for these apparatuses. The bag for these apparatuses generally has a maximum volume of less than one liter. The weight of a full apparatus is therefore always less than 1 kg. The force applied to check the tightness seal and hermeticity of the apparatus should therefore be at least two times more than the force corresponding to this weight, or at least 20 N. A static tightness seal test is currently used, whereby a force of 200 N is applied to an apparatus that is full of water and set horizontally.

Before describing the connector for an ostomy apparatus according to the invention in operating position, its two members are considered separately.

The first member 10 of the connector includes a flap 12 intended to be permanently held by a rubber washer glued to the skin of a user. This flap 12 surrounds a central opening 13 represented in the shape of a circle.

A flange 14, separate from the flap 12, defines with the flap a groove 16 radially turned outward. Preferably, the flange 14 is wider at various locations of its periphery, particularly in the portion that is intended to be located at the bottom when the user is standing, as shown in reference 18, and two locations 20 separated by around 140° from the portion of the flange 14 intended to be located at the top when the user is standing.

The first member 10 has good flexibility, allowing it to adapt to the curvature of the user's skin. It can thus be made from an olefin and a vinyl acetate copolymer elastomer, such as ethylene or propylene.

The second member 22 includes, around a central opening 23, an elastic ring 24 attached to a flap 26. The elastic ring 24 preferably has a circular section, and the inside diameter of the opening 23 defined by the elastic ring at rest is less than the diameter of the groove 16 of the first member.

The second member 22 also includes, between the flap 26 and the elastic ring 24, a deformable area 28 represented in the form of a U-shaped fold on one side of the flap 16. More specifically, the deformable area 28 extends radially around the elastic ring 24, connecting the outside periphery of said ring 28, facing outwards from the opening 23, to the inside periphery of the flap 26, facing the opening 23.

This deformable area constitutes the device for applying the weight of the apparatus to the only portion of the elastic ring located above the groove according to the invention, in the considered embodiment. However, this device may have many other forms. For example, it can be formed of a deformable area with two consecutive folds that are adjacent or in a Z shape, one turned toward inside and the other toward the outside.

In one variant, this deformable area may also extend only to a portion of the outside periphery of the elastic ring 24, corresponding to the lower portion on the standing user. In this case, in the portion of the outside periphery of the elastic ring 24 without a deformable area, said ring 24 is attached directly to the inside periphery of the flab 26.

In addition, the second member preferably includes three pins 30′ 30″ 30′″ that protrude radially toward the outside of the elastic ring 24 to which they are attached. (They can also be attached to the portion of the flap located between the elastic ring 24 and the deformable area 28).

Although the pins 30′ 30″ 30′″ are represented as protruding from the periphery of the flap 26, they can also not protrude from it.

The role of the pin 30′ 30″ 30′″ is to allow the user to grab hold of it to stretch the elastic ring 24 when placing it into the groove of the first member 10 or when removing it from this groove, as described in more detail later.

The second member 22 may be formed by injecting a plastic material, for example with an ethylene vinyl acetate copolymer, or preferably with a styrene-ethylene/butylenes-styrene or styrene-isoprene-styrene copolymer.

Before describing the use of the connector, the cooperation of the two members is considered, with reference to FIG. 6.

In FIG. 6, a strap-like holder is made of a rubber washer 32 glued to the skin 31 of the user and having a central opening 33 in which the ostomy is located. The first member 10 of the connector has its flap 12 permanently attached, by glue in this case, to the rubber washer 32. The holder is therefore formed entirely by the rubber washer 32 and the first member 14 of the connector. The opening 13 of the first member of the connector corresponds to the opening 33 of the rubber washer.

The peripheral portion of the flap 26 of the second member 22 is tightly soldered in 36 along an opening in a piece of foil 34 on the bag, and its elastic ring 24 is lodged into the groove of the first connector member.

Assuming that the user wants a redundant attachment device, a possible attachment device 38 was also shown schematically, with loops and hooks, attached between the flap 12 of the first member, which may then be extended locally as shown on the right in FIG. 6, and the outside part of the flap 26 of the second member. This device 38 can also represent snaps or any other suitable device. FIG. 8, described later, represents a variant of the redundant attachment device. However, it is not at all necessary according to the invention, except to give the user a feeling of double security.

When setting up an apparatus, the holder formed from the rubber washer 32 and the first member 10 is already glued to the user's skin in the position indicated in FIG. 1, with the enlargement 18 of the flange 14 facing downward.

The user grabs hold of the apparatus by the two pins 30″ 30′″ of the second member 22 of the connector and places the lower portion of the elastic ring 24 under the enlarged portion 18. By putting upward, the user flattens this lower portion of the elastic ring against the groove of the lower portion. Then the user pulls the pins 30″ 30′″ upward to stretch the plastic ring 24 and make it go above the two enlargements 20 of the flange 14. Finally, the user grabs hold of the upper pin 30′ and pulls it upward to bring the corresponding portion of the elastic ring 24 into the groove 16 by releasing the pin 30′. The elastic ring 24, whose inside diameter is less than that of the groove 16, is elastically flattened against the groove all around it. The seal is thus formed.

As the bag gradually fills, the weight applied to the flap 26 increases. The outside portion of the flap 26 therefore tends to move increasingly more downward. At this time, the deformable area 28 compresses the upper portion and moves away from the lower portion. Given the V, U or Z shape of the deformable area 28, the upper portion of the flap 26 is compressed and supports the weight of the apparatus before the lower portion is stretched enough to transmit a substantial force to the lower portion of the elastic ring 24.

Consequently, the weight it transmitted by the flap 26 to the upper portion of the elastic ring 24, as a compressive force of the elastic ring against the groove, without practically any force moving the lower portion of the elastic ring 24 from the groove.

When the bag must be removed from the holder, the user first grabs hold of the upper pin 30′ and pulls it upward so that the corresponding portion of the elastic ring 24 is outside of the groove 16. Then the user grabs hold of the side pins 30″ and 30′″ to gradually disengage the rest from the elastic ring 24, over the enlargements 20 of the groove 16, and then lower the bag to remove and empty it.

Although the flap 26 is equipped with a deformable area 28 extending along its outer periphery, this deformable area may be present only in some locations, such as on the lower outer periphery of the flap 26. In addition, it is conceivable for there to be other deformable areas. For example, the thickness of the flap 26 can be increased on the top portion or along a preferred path for transmitting the apparatus weight. However, such a solution is not necessarily comfortable for the user, since the flap 26 may become too hard locally.

FIG. 7, which is similar to FIG. 1, represents a variant of the first member of a connector of an ostomy apparatus according to the invention, with identical references to those in FIG. 1 designating similar elements.

In this variant, the flange 14 of the apparatus in FIGS. 1 through 6 comprises not only the enlargements 18, 20, but also the enlargements 40 pointing upward.

When the user sets up the apparatus by pulling the elastic ring by the pins, he can pause midway, when the elastic ring has gone past the enlargements 40 but has not yet reached the enlargements 20. For example, the user may pull a pin 30″ 30′″ from one side until the ring goes past the enlargement 40 on that side and the possibly release said pin 30″ 30′″ on that side and grab hold of a pin 30″ 30′″ on the opposite side to bring it past the other enlargement.

It is thus possible to set up or remove the apparatus using only one hand. Even though a single enlargement 40 was represented on each side, it is possible to incorporate several of them, which then forms a sort of rack.

FIG. 8, which is similar to FIG. 3, represents a variant of the second member of a connector of an ostomy apparatus according to the invention, with identical references to those in FIG. 3 designating similar elements.

In FIG. 8, a second member of the connector comprises a flap 42 that includes ears 44 at its periphery. These ears 44 each have a hole 46 intended for a rivet (not represented) from a first member, similar to that in FIG. 1, whose flap 12 has such rivets. When the user places the elastic ring 24 in the groove 16, he may introduce one or more rivets into one or more holes in the flap 42. The connector then cannot be opened as long as the rivets are not removed from the corresponding holes.

Although a connector is represented with an elastic ring 24 having three traction pins 30′ 30″ 30′″, it is possible to use only one pin facing upward or two pins separated by 90°. Likewise, it is possible to have more than three pins so that the user may choose the pins considered to be the most convenient for setting up or extracting the apparatus.

1. A connector for an ostomy apparatus of the type that includes a first member attached to a holder to be attached to the skin of a user and a second member attached to a bag that forms a receptacle.
the first member defines an opening and is made of a flexible material so that it can adapt to variations in the shape of the part of the user's body to which the holder is attached, and it defines a continuous groove emerging radially around the opening, and
the second member includes an elastic ring surrounding an opening and a flap globally disposed on a plane between the elastic ring and the periphery thereof, with the at-rest inside diameter of the second member's elastic ring being less than the diameter of the first member's groove and the flap being designed to be secured to a foil of a bag,
wherein the second member also includes a device for applying the weight of the apparatus to the only portion of the elastic ring located above the groove, said device for applying the weight of the apparatus being formed from a deformable area extending between the ring and the flap, and on at least a partial perimeter of the ring, said deformable area having at rest a V, U or Z-shaped fold in order that said deformable area being capable of being compressed in the upper portion and being stretched in the lower portion of the ring when the weight of the bag increases, the compressive force of the elastic ring against the upper portion of the groove being in in such a way that the lower portion of the elastic ring does not tend to move away from the lower part of the groove.

2. A connector according to claim 1, wherein the deformable uncoupling area is such that a portion of the flap adjacent to the elastic ring and a portion of the flap adjacent to the periphery allow a relative movement on said plane when the flap is subjected to a force acting on its plane.

3. A connector according to claim 1, wherein the deformable area has its greatest deformation capacity on the side intended to be located at the bottom on a standing user.

4. A connector according to claim 1, wherein the second member has a least one traction pin for applying a radial outward force to the elastic ring.

5. A connector according to claim 4, wherein the pin is attached at a location that is closer than the deformable area to the inside of the flap.

6. A connector according to claim 4, wherein the second member includes three traction pins spaced about 13° apart and located opposite of the side intended to be located at the bottom on a standing user.

7. A connector according to claim 1, wherein the second member formed by the elastic ring, the flap, the deformable area, and the pins, if applicable, is formed in one piece.

8. A connector according to claim 1, wherein the groove of the first member is defined, on the side intended to be farthest from the holder, by an outer flange whose radial dimension on the outside of the opening is greater than the side intended to be located at the bottom on a standing user.

9. A connector according to claim 8, wherein the radial dimension of the first member's outer flange is greater at each location that corresponds to a pin on the second member.

10. An ostomy apparatus of the type that comprises at least two layers of foil defining a receptacle, wherein one foil is attached to a member constituted by a second member of a connector according to claim 1.
